(19) United States
(12) Patent Application Publication
(10) Pub. No.: US 2009/0143826 A1
(43) Pub. Date: Jun. 4, 2009

(54) FASTENING SYSTEM AND FASTENING METHOD FOR REFERENCE ARRAYS

(76) Inventors: Rainer BIRKENBACH, Erdlng (DE); Martin Immerz, Grafelfing (DE); Manuel Millahn, Munchen (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/326,923

(22) Filed: Dec. 3, 2008

Related U.S. Application Data
(60) Provisional application No. 60/992,454, filed on Dec. 5, 2007.

(30) Foreign Application Priority Data
Dec. 3, 2007 (EP) 07 122 134 5

(51) Int. Cl. A61B 17/04; A61B 17/56

(52) U.S. Cl. 606/301; 606/60

(57) ABSTRACT

A fastening system for reference arrays, wherein a curable material fixes an attachment base which is assigned to the reference array, relative to a bone.

### RELATED APPLICATION DATA

This application claims the priority of U.S. Provisional Application No. 60/992,454, filed on Dec. 5, 2007, which is hereby incorporated in its entirety by reference.

### FIELD OF THE INVENTION

The invention relates to a fastening system and/or fastening method for reference arrays which are attached to a bone, in order to enable it to be located and tracked with the aid of a medical navigation and/or tracking system.

### BACKGROUND OF THE INVENTION

Such reference arrays have previously been attached to the bone to be tracked using screw connections. The screws used for this purpose comprise a self-cutting thread which cuts into the bone substance when the screw is screwed in, wherein no hole is drilled into the bone substance beforehand, such that the bone is ultimately exposed to high mechanical loads when the reference arrays are attached. This is in particular problematic in the case of older patients, whose bones do not exhibit a correspondingly high load limit. If the reference array is bicortically attached, wherein the bone cortex is penetrated at two locations—in most cases, two proximate locations—on the bone by attaching a holding device for the reference array, the bone structure can still be weakened such that post-operative fractures or intra-operative fractures while introducing the implant may occur. Thus, while fastening the reference arrays using screws, bone fragments may break off at the location of the screw connection or, in the worst case scenario, the bone may break at this location.

### SUMMARY OF THE INVENTION

It is the object of the invention to provide a fastening system and/or fastening method for reference arrays, using which reference arrays can be attached to the bone in a minimally damaging way.

This object is solved by a fastening system for reference arrays, wherein a curable material fixes an attachment base which is assigned to the reference array, relative to a bone, and by a fastening method for fastening a reference array to a bone using a curable material which fixes an attachment base which is assigned to the reference array, relative to a bone, comprising at least one of the following steps: placing the attachment base into or onto a bone, in particular into or onto a receptacle which has been formed, especially onto the processed surface of the bone or into a blind hole drilled in the bone beforehand; plasticising the material, in particular by at least one of the following influences which act on the material: ultrasonic waves; thermal energy; infrared radiation; microwave radiation; UV radiation; conveying the viscous material, in particular through a channel arranged in the attachment base, into a region which abuts the attachment base and the bone; hardening the material; removing the attachment base from the bone. The sub-claims define preferred embodiments of the invention.

In the fastening system in accordance with the invention, an attachment base which is assigned to a reference array, i.e. can be fastened to it, is fixed relative to the bone by means of a curable material, wherein it is possible to distinguish primarily between materials which are merely curable and materials which can additionally be re-plasticised. In the case of the latter, it would therefore be possible in principle to repeatedly cure and then re-plasticise the material. The material is thus a kind of bonding agent which establishes the force fit between the bone and the attachment base. In other words, the attachment base is bonded to the bone. The following materials may be considered for this purpose: polymers, polymer-ceramics, cement, metal, glass, bone cement, ceramics (Al₂O₃, TiO₂, ZrO₂), metal powders (Ti, Fe) and glass (SiO₂).

In a preferred embodiment, the curable material is initially provided in a viscous state and is cured or hardened as soon as it has been conveyed into the region where the “bond” between the bone and the attachment base is to be established. This region is generally situated between the attachment base and the bone, wherein the material can be hardened automatically, i.e. without external influences, or only once external influencing factors have been applied to the material, wherein heat applied from without, UV radiation or the like would for example be conceivable. Once cured, the material is stable and/or resilient, such that the attachment base and therefore also the reference array is fixed relative to the bone and can no longer move relative to the bone.

It is in principle also conceivable for a bone fragment belonging to the bone to be fixed to the bone instead of an attachment base of reference arrays, such that for example after a bone fracture, the bone fragments are “bonded” to each other.

It is also conceivable for the material to be provided in a part of the attachment base, for example a reservoir of the attachment base, before it is conveyed into the region between the attachment base and the bone where it is ultimately hardened. It is thus not necessary to provide any material externally, in addition to the attachment base, which substantially simplifies manipulating the fastening system. Once the attachment base is situated at a desired location on the bone, the material can for example be output from a reservoir of the attachment base, in order to ultimately be cured in the region between the attachment base and the bone, wherein the material can for example be pressed out of the attachment base by a piston or, if it is provided in the attachment base in a solid state, can be plasticised and can run out of the attachment base or can be pressed out of the attachment base, in order to ultimately be cured in the region between the attachment base and the bone.

It is however also conceivable for the material to be introduced into the region between the attachment base and the bone “from without”, for example using a kind of bonding instrument which, when activated, releases the material.

In accordance with a preferred embodiment, the curable material can additionally be plasticised by an external influence, as soon as the latter is applied to the material. Thus, for example, the material can be plasticised by ultrasonic waves, thermal energy, infrared radiation, microwave radiation or UV radiation, as soon as these are applied to the material, for example using a separate apparatus.

Furthermore, two types of an attachment base are in principle conceivable.

In the first type, the attachment base is merely placed onto the bone, wherein the material is arranged between the surface of the bone and the attachment base. In order to improve the grip of the material on the surface of the bone, the surface of the bone can be roughened by suitable means before the attachment base is placed on it. Thus, it is not necessary to further process the bone substance. This can be particularly advantageous for bones which are in danger of breaking even under small mechanical loads.

In the second type of attachment base, the latter protrudes through the bone cortex, into the bone spongiosa. This is in particular achieved by forming a recess in the bone, for example drilling a blind hole in the bone, into which the attachment base is inserted, wherein the material can be introduced into the region between the attachment base and the bone before or after the attachment base is inserted, such that the attachment base is completely surrounded in the bone by curable material. This achieves an additional stability of the “bond”, since the curable material can also penetrate into the porous bone spongiosa and connect to it in a positive fit.

Since the attachment base terminates in the spongiosa and only penetrates the bone cortex at one location, this is referred to as unicortically attaching the attachment base. Contrary to the present invention, reference arrays in the prior art are often attached bicortically, wherein the bone cortex is penetrated by parts of the reference array on two opposing sides of the bone. While this does lead to an increased load on the bone tissue, it was however necessary, since a unicortical screw connection is in danger of detaching during use. Due to the positive-fit connection between the attachment base and regions of the spongiosa, this is more resilient and there is therefore no need to worry about the attachment base coming loose from the bone.

In another embodiment, however, it is conceivable for the material to only be provided in a base region of the blind hole in the bone, such that after it has been used, the attachment base can be easily detached again from the bone.

It is also conceivable for the attachment base to comprise a channel through which the material can be conveyed into the region between the attachment base and the bone, through at least one output opening. This can in particular be envisaged in conjunction with a reservoir for the material in the attachment base, since such an embodiment substantially facilitates manipulating the attachment base, wherein it is also conceivable for the channel to comprise protrusions or cavities on its interior wall within the attachment base. If the material is cured both in the region between the attachment base and the bone, and through the output opening of the channel into the channel, a positive fit between the attachment base and the bone can be ensured by the protrusions or cavities on the interior wall of the channel. If the attachment base does not then experience any bond through the material except within the channel (for example, due to a smooth exterior wall of the attachment base), the attachment base can be detached from the bone after it has been used by removing the material within the channel. Thus, in the case of a cylindrical channel comprising protrusions or cavities, the attachment base can be detached from the bone by drilling out the material from the attachment base.

Protrusions or cavities can, however, also be formed on the exterior wall of the attachment base, which generate a positive fit between the attachment base and the bone, via the material.

It is also possible to configure the attachment base such that the reference array can be removed from the attachment base. The attachment base can thus be attached to the bone and, when on it, detached again independently of the reference array. The reference array is only attached to the attachment base for use in conjunction with a navigation and/or tracking system.

The invention also relates to a method for fastening a reference array to a bone using a curable material, wherein the attachment base is placed in or on a bone as already described above, wherein the material can already be provided in a fluid state or is firstly plasticised by ultrasound, thermal energy, infrared radiation, microwave radiation or UV radiation, in order to be conveyed into a region between the attachment base and the bone. The material for the region can also be supplied via a channel formed in the attachment base. The material is then hardened, either by external influences or automatically, and thus fixes the attachment base to the bone. Once the reference array which is attached to the attachment base has fulfilled its purpose in conjunction with a navigation and/or tracking system, the attachment base can be removed from the bone again. This can be achieved on the one hand by re-plasticising the material, or by mechanically removing the material, for example by drilling the material out from the supply channel in the attachment base, wherein the material can be re-plasticised by the same influences as have already been described above. It is also conceivable for the attachment base to partially remain in or on the bone, wherein the protruding and/or interfering part of the attachment base is mechanically severed, such that the remaining relic of the attachment base ends flush with the bone. This can in particular be achieved in conjunction with an attachment base which consists of a biologically degradable material and is degraded and/or grows into the bone in the course of time. However, metal or bone tissue are also conceivable as the material for the attachment base.

### BRIEF DESCRIPTION OF THE DRAWINGS

The invention is illustrated in more detail in individual embodiments, on the basis of the enclosed figures, wherein it can comprise the features described here, individually and in any expedient combination. It is conceivable for an attachment base made of a suitable material (for example, metal) to both be formed as a tool and/or drill for forming a blind hole in the bone and to simultaneously also comprise features described here. The attachment base can thus simultaneously be used to drill the blind hole and to fix the reference array.

FIG. 1 shows an attachment base comprising a structured exterior surface.

FIG. 2 shows an attachment base comprising a supply channel and output openings for the material.

FIG. 3 shows an attachment base comprising a structured interior wall of the supply channel.

FIG. 4 shows a perspective view of an attachment base.

FIG. 5 shows an attachment base which ends flush with the surface and comprises a supply channel and an output opening.

FIG. 6 shows an attachment base for placing onto the surface of the bone, comprising a plurality of supply channels.

FIG. 7 shows an attachment base for placing onto the surface of the bone, comprising one supply channel.

FIG. 8 shows an attachment base, embedded in material, on the surface of the bone.

FIG. 9 shows an attachment base with a reference array fastened to it.

FIG. 10 shows an attachment base with a reference array detached from it.

### DETAILED DESCRIPTION

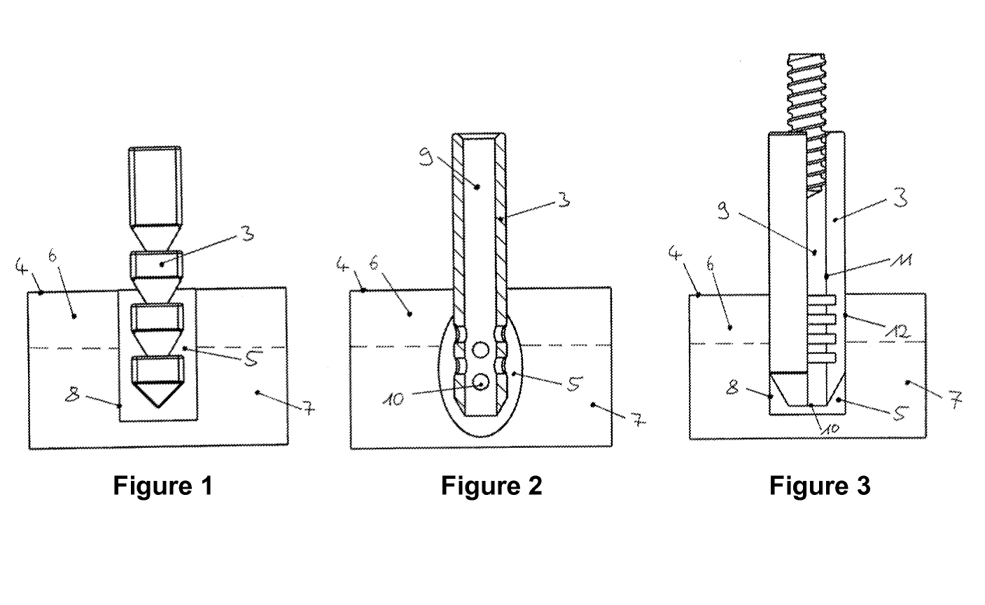

FIG. 1 shows an attachment base 3 which is inserted into a blind hole 8 drilled in a bone 4. The attachment base 3 extends together with the blind hole 8 through the bone cortex 6, into the bone spongiosa 7, wherein the blind hole 8 exhibits a slightly larger diameter than the attachment base 3, such that a region 5 is created, into which the material can be introduced, wherein the attachment base 3 exhibits a structured surface in the form of radially circumferential grooves which ensure the positive fit between the bone 4 and the attachment base 3.

FIG. 2 shows an attachment base 3 which comprises a channel 9, through which the material can be conveyed into the region 5. For this purpose, the end of the attachment base 3 positioned in the bone 4 comprises openings 10 through which the material can be output. The region 5 thus extends into the bone spongiosa 7, wherein a positive fit is realized by material provided in the bone spongiosa 7 and in the output openings 10.

A separately structured exterior surface of the attachment base 3 is therefore also not necessary.

FIG. 3 shows an attachment base 3 in which the exterior diameter is the same size as that of the blind hole 8. The attachment base 3 thus directly abuts the bone 4, such that the material can only be provided in the region 5 in the lower region of the blind hole 8, but extends through the output opening 10 into the channel 9 of the attachment base, wherein the channel 9 comprises a plurality of radially circumferential grooves which ensure the positive fit between the attachment base 3 and the bone 4. A drilling instrument is additionally shown at the upper end of the attachment base 3, and can be inserted into the channel 9 from above and mechanically removes the material, in order to detach the attachment base 3. Once the circumferential grooves on the interior wall of the channel 9 have been cleared of material, the attachment base 3 can be removed from the bone 4.

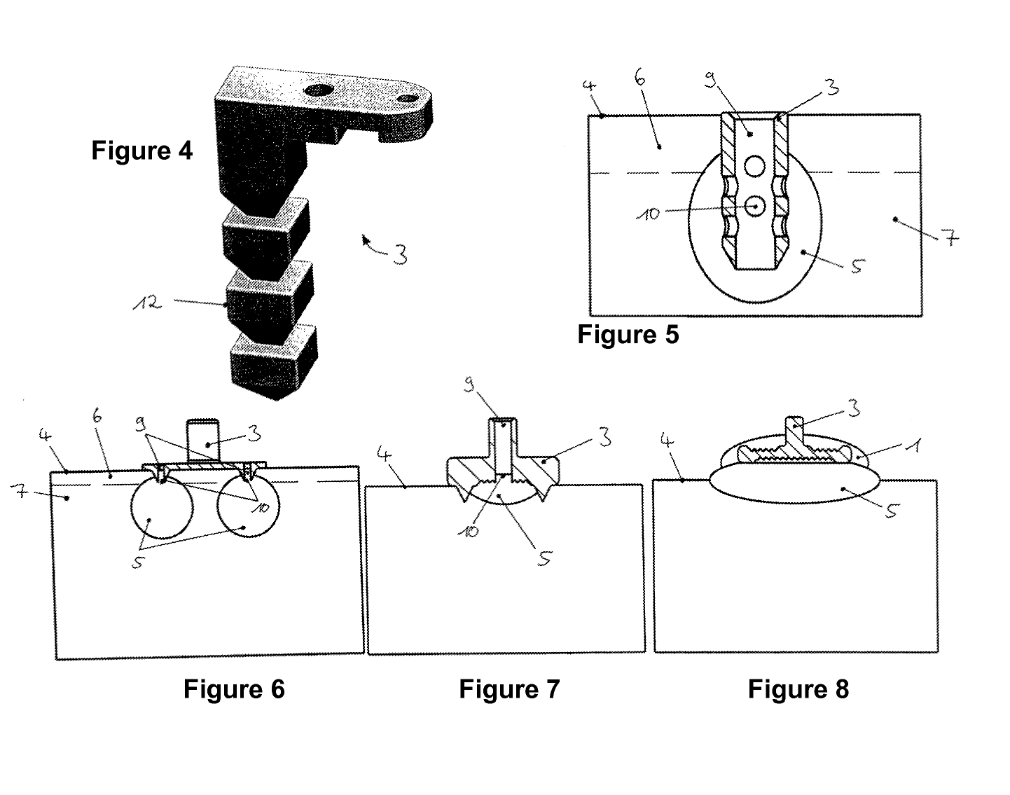

FIG. 4 shows an attachment base 3, similar to the attachment base 3 from FIG. 1, which additionally comprises a receptacle for a reference array on its upper end. The reference array 2 can thus be detached from the attachment base 3.

FIG. 5 shows an attachment base 3 which ends flush with the surface of the bone 4 and exhibits the same features as the attachment base 3 from FIG. 2. However, this attachment base is shortened, such that it can remain in the bone 4 after it has been used, since it does not form an interfering protrusion on the bone 4.

FIG. 6 shows an attachment base 3 which is merely placed onto the surface of the bone 4. In a disc-shaped portion, a plurality of channels 9 are formed, through which the material 1 can be conveyed into the regions 5, so as to form a positive fit between the attachment base 3 and the bone 4, wherein continuations extend together with the channels 9 and the openings 10 through the bone cortex 6, into the spongiosa 7, such that the material is arranged in the spongiosa 7 once it has been conveyed.

FIG. 7 shows an attachment base 3 for placing onto a surface of a bone 4, wherein the underside of the attachment base 3 is structured, in order to ensure a secure positive fit between the attachment base 3 and the bone 4, wherein the material is conveyed through the channel 9 of the attachment base 3, out of the opening 10, into the region 5 between the attachment base 3 and the bone 4. Thus, the material is only provided between the attachment base 3 and the bone 4, and does not penetrate into the spongiosa 7.

FIG. 8 shows an attachment base 3 which is placed onto a region 5 on the surface of the bone 4 which has been charged with material beforehand, and is fixed to the bone 4 by subsequently applying additional material 1 to the region 5.

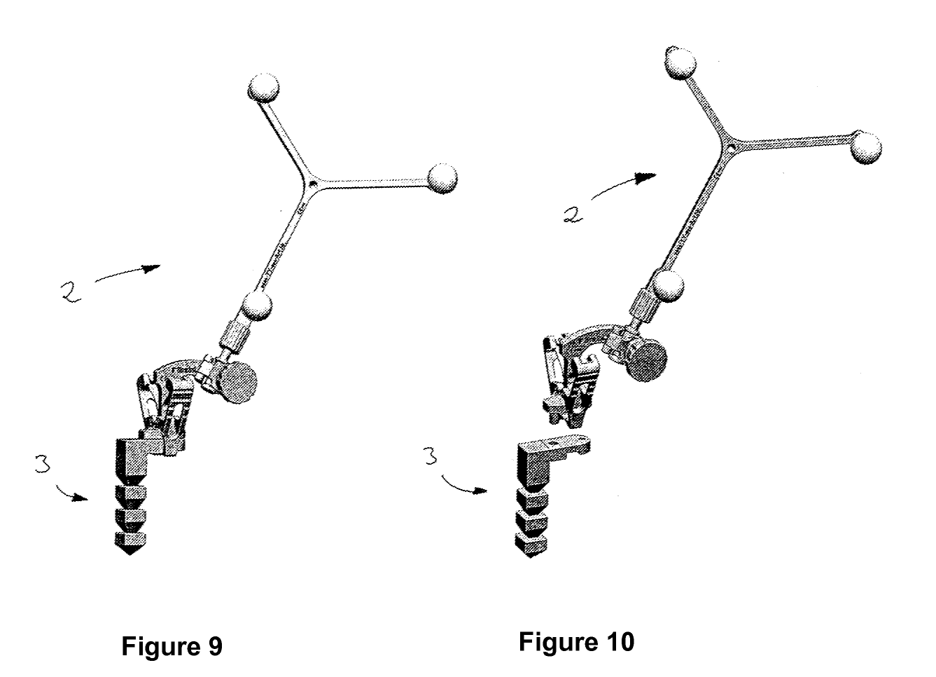

FIG. 9 shows a perspective view of an attachment base 3 from FIG. 1 and/or FIG. 4, wherein a reference array 2 comprising three reflectors is fastened to the upper end of the attachment base 3, wherein the position and alignment of the reference array 2 with respect to the attachment base 3 can be varied by an adjustable connecting member arranged between the attachment base 3 and the reference array 2.

FIG. 10 shows the reference array 2 and attachment base 3 from FIG. 9, wherein the reference array 2 is detached from the attachment base 3.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiments.

Although the invention has been shown and described with respect to one or more particular preferred embodiments, it is clear that equivalent amendments or modifications will occur to the person skilled in the art when reading and interpreting the text and enclosed drawings of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment or embodiments illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A fastening system for reference arrays, wherein a curable material fixes an attachment base which is assigned to the reference array, relative to a bone.

2. The fastening system for reference arrays in accordance with claim 1, wherein the curable material is provided in a viscous form while it is being conveyed into a region which abuts the attachment base and the bone and—once it comes to rest in the region—assumes a solid state, in order to fix the attachment base relative to the bone.

3. The fastening system for reference arrays in accordance with claim 2, wherein the solid state is a stable state.

4. The fastening system for reference arrays in accordance with claim 1, wherein the material is provided in a part of the attachment base before being conveyed into the region.

5. The fastening system for reference arrays in accordance with claim 1, wherein the material is provided outside the attachment base before being conveyed into the region.

6. The fastening system for reference arrays in accordance with claim 1, wherein the material can be plasticised by an external factor applied to the material.

7. The fastening system for reference arrays in accordance with claim 6, wherein the external factor is one or more of the following:
ultrasonic waves;
thermal energy;
infrared radiation;
microwave radiation;
UV radiation.

8. The fastening system for reference arrays in accordance with claim 1, wherein the attachment base is placed onto the bone, and the region which abuts the attachment base and the bone is substantially arranged on a part of the exterior side of a bone cortex.

9. The fastening system for reference arrays in accordance with claim 1, wherein the attachment base extends through the bone cortex, into the bone spongiosa, and the region which abuts the attachment base and the bone is arranged around the attachment base.

10. The fastening system for reference arrays in accordance with claim 9, wherein the attachment base extends into a blind hole drilled in the bone beforehand.

11. The fastening system for reference arrays in accordance with claim 9, wherein the region which abuts the attachment base and the bone is a base region of the blind hole drilled in the bone beforehand.

12. The fastening system for reference arrays in accordance with claim 1, wherein a channel extends through the attachment base, wherein at least one output opening is connected to the channel, in order to guide the viscous material to the region in which it is then cured.

13. The fastening system for reference arrays in accordance with claim 12, wherein the channel extends along a longitudinal axis of the attachment base.

14. The fastening system for reference arrays in accordance with claim 12, wherein the interior wall of the channel comprises at least one cavity or constriction, and the exterior wall of the attachment base exhibits a substantially smooth surface.

15. The fastening system for reference arrays in accordance with claim 14, wherein the at least one cavity or constriction is at least one radially circumferential groove in the interior wall.

16. The fastening system for reference arrays in accordance with claim 1, wherein the exterior wall of the attachment base comprises at least one cavity, constriction or elevation.

17. The fastening system for reference arrays in accordance with claim 16, wherein the at least one cavity, constriction or elevation is at least one radially circumferential groove or collar on the exterior wall.

18. The fastening system for reference arrays in accordance with claim 1, wherein the viscous material extends at least partially into the bone spongiosa before it is cured.

19. The fastening system for reference arrays in accordance with claim 1, wherein the reference array can be removed from the attachment base.

20. A navigation system comprising at least one reference array which is fixed to a bone in accordance with claim 1.

21. A method for fastening a reference array to a bone using a curable material which fixes an attachment base which is assigned to the reference array, relative to a bone, comprising at least one of the following steps:
placing the attachment base into or onto a bone;
plasticising the material;
conveying the viscous material;
hardening the material;
removing the attachment base from the bone.

22. The method in accordance with claim 21, wherein the attachment base is placed into or onto a receptacle which has been formed.

23. The method in accordance with claim 21, wherein the attachment base is placed onto the processed surface of the bone or into a blind hole drilled in the bone beforehand.

24. The method in accordance with claim 21, wherein the material is plasticised by at least one of the following influences which act on the material:
ultrasonic waves;
thermal energy;
infrared radiation;
microwave radiation;
UV radiation.

25. The method in accordance with claim 21, wherein the viscous material is conveyed through a channel arranged in the attachment base, into a region which abuts the attachment base and the bone.

26. The method in accordance with claim 21, wherein in order to remove the attachment base, the material is re-plasticised.

27. The method in accordance with claim 26, wherein the material is re-plasticised by at least one of the following factors which act on the material:
ultrasonic waves;
thermal energy;
infrared radiation;
microwave radiation;
UV radiation.

28. The method in accordance with claim 21, wherein in order to remove the attachment base, the attachment base is mechanically severed.

29. The method in accordance with claim 28, wherein the attachment base is mechanically severed such that the part of the attachment base remaining on the bone ends flush with the bone.

30. The method in accordance with claim 21, wherein in order to remove the attachment base, at least some of the material is mechanically removed.

31. The method in accordance with claim 30, wherein the material is mechanically removed by drilling out a round supply channel in the attachment base.

32. A computer program stored on a machine-readable medium for fastening a reference array to a bone using a curable material which fixes an attachment base which is assigned to the reference array, relative to a bone, comprising:
code for placing the attachment base into or onto a bone;
code for plasticising the material;
code for conveying the viscous material;
code for hardening the material;
code for removing the attachment base from the bone.

33. The computer program in accordance with claim 32, wherein the attachment base is placed into or onto a receptacle which has been formed.

34. The computer program in accordance with claim 32, wherein the attachment base is placed onto the processed surface of the bone or into a blind hole drilled in the bone beforehand.

35. The computer program in accordance with claim 32, wherein the material is plasticised by at least one of the following influences which act on the material:
ultrasonic waves;
thermal energy;
infrared radiation;
microwave radiation;
UV radiation.

36. The computer program in accordance with claim 32, wherein the viscous material is conveyed through a channel arranged in the attachment base, into a region which abuts the attachment base and the bone.
